(19) United States
(12) Patent Application Publication
(10) Pub. No.: US 2025/0049840 A1
(43) Pub. Date: Feb. 13, 2025

(54) COMPOSITIONS COMPRISING A CHEMOKINE RECEPTOR PATHWAY INHIBITOR

(71) Applicant: DIMERIX BIOSCIENCE PTY LTD, Fitzroy, Victoria (AU)

(72) Inventors: Bronwyn Jane POLLOCK, Blackburn South (AU); Robert SHEPHERD, Upwey (AU); Nina WEBSTER, Strathmore (AU)

(73) Assignee: DIMERIX BIOSCIENCE PTY LTD, Fitzroy, Victoria (AU)

(21) Appl. No.: 18/718,617

(22) PCT Filed: Jan. 14, 2022
(86) PCT No.: PCT/AU2022/050013

§ 371 Date: Jun. 11, 2024

(51) Int. Cl. A61K 31/80; A61K 9/20; A61K 9/28; A61K 9/48

(52) U.S. Cl. CPC A61K 31/80; A61K 9/2009; A61K 9/2013; A61K 9/2054; A61K 9/2059; A61K 9/28; A61K 9/4858; A61K 9/4866

(57) ABSTRACT

A composition comprising: i. at least one chemokine receptor 2 (CCR2) pathway inhibitor or a pharmaceutically acceptable salt thereof, in particular repagermanium or propagermanium: ii. a diluent in a concentration from 30 to 70% w/w; iii. a binder in a concentration from 2 to 5% w/w: iv. a disintegrant in a concentration from 2 to 8% w/w; and v. a lubricant in a concentration from 0.05 to 5% w/w; and methods of treatment with same.

### FIELD OF THE INVENTION

The present invention relates to a composition comprising a chemokine receptor pathway inhibitor and methods of using the same.

### BACKGROUND ART

Therapies for diseases such as kidney disease and respiratory disease based on a chemokine receptor 2 (CCR2) pathway inhibitor are known.

Repagermanium or propagermanium may be used as a CCR2 pathway inhibitor. However, whilst propagermanium was approved in Japan in the 1990s for treating hepatitis B, it has not been approved elsewhere and is classified as a New Chemical Entity (NCE). Repagermanium and propagermanium have previously been described in the literature as polymeric compounds. The drug substance 3-[(2-carboxyethyl-oxogermyl)oxy-oxogermyl] propanoic acid exhibits polymorphism and can exist in different solid forms. However, the present inventors have not observed any pharmacological effects of polymerisation of repagermanium during manufacture or testing of the drug substance.

Polymorphs of 3-[(2-carboxyethyl-oxogermyl)oxy-oxogermyl] propanoic acid include repagermanium, propagermanium, proxigermanium, Ge-132, germanium sesquioxide, 2-carboxyethylgermasesquioxane, SK-818, bis (2carboxyethylgermanium) sesquioxide.

The two most highly characterised solid forms, propagermanium and repagermanium share a common core structure (Mizuno, et al, 2015). These two drug substances have different International Nonproprietary Names (INNs), the INN for propagermanium being INN #6260 and repagermanium being INN #6259. They are comprised of different packing of the monomeric molecule 3-[(2-carboxyethyl-oxogermyl)oxy-oxogermyl] propanoic acid, varying in the structural units coordinated by H bonds. X-ray diffraction has revealed that propagermanium has a polymeric ladder-shaped structure of a concatenated eight-membered ring composed of Ge-O bonds, which is clearly distinguished from the infinite sheet structure in repagermanium. In solid form, propagermanium and repagermanium differ; however, in solution they are identical as the structural units are dissolved.

The inventors have surprisingly found a novel composition for the delivery of a CCR2 blocker which overcomes some or all of the shortcomings of the prior art or at least provides a commercial alternative. The preceding discussion is intended only to facilitate an understanding of the invention. It should not be construed as in any way limiting the scope or application of the following description of the invention, nor should it be construed as an admission that any of the information discussed was within the common general knowledge of the person skilled in the appropriate art at the priority date.

### SUMMARY OF THE INVENTION

The present invention provides a composition comprising:
i. at least one chemokine receptor 2 (CCR2) pathway inhibitor or a pharmaceutically acceptable salt thereof;
ii. a diluent in a concentration from 30% to 70% w/w;
iii. a binder in a concentration from 2% to 5% w/w;
iv. a disintegrant in a concentration from 2% to 8% w/w; and
v. a lubricant in a concentration from 0.05% to 5% w/w.

Preferably the composition comprises from 2.5% to 65% w/w of the CCR2 pathway inhibitor. Preferably the CCR2 pathway inhibitor is a direct antagonist of CCR2. More preferably the CCR2 pathway inhibitor is repagermanium or propagermanium or a pharmaceutically acceptable salt thereof.

Preferably the diluent is silicified microcrystalline cellulose (SMCC). Preferably the composition comprises from 30% to 70% w/w SMCC.

Preferably the binder is hypromellose (HPMC). Preferably the composition comprises from 2% to 5% w/w HPMC.

Preferably the disintegrant is sodium starch glycolate. Preferably the composition comprises from 2% to 8% w/w sodium starch glycolate.

Preferably the lubricant is magnesium stearate. Preferably the composition comprises from 0.05% to 5% w/w magnesium stearate.

The present invention further provides a method for the treatment, amelioration or prevention of a condition or disease comprising administering to said subject a therapeutically effective amount of:
a) a composition comprising
   i. at least one chemokine receptor 2 (CCR2) pathway inhibitor or a pharmaceutically acceptable salt thereof;
   ii. a diluent in a concentration in a concentration from 30% to 70% w/w;
   iii. a binder in a concentration in a concentration from 2% to 5% w/w;
   iv. a disintegrant in a concentration in a concentration from 2% to 8% w/w; and
   v. a lubricant in a concentration in a concentration from 0.05% to 5% w/w.

Preferably the method comprises administering from 10 mg to 500 mg per day of the CCR2 pathway inhibitor, provided in one or more doses.

Preferably the condition or disease to be treated, ameliorated or prevented is chosen from: kidney disease, cardiovascular disease, eye disease, oncological disease, or pulmonary disease.

Use of:
i. at least one chemokine receptor 2 (CCR2) pathway inhibitor or a pharmaceutically acceptable salt thereof;
ii. a diluent in a concentration from 30% to 70% w/w;
iii. a binder in a concentration from 2% to 5% w/w;
iv. a disintegrant in a concentration from 2% to 8% w/w; and
V. a lubricant in a concentration from 0.05% to 5% w/w.

in the manufacture of a medicament for the treatment, amelioration or prevention of a condition or disease.

Use of a composition comprising:
i. at least one chemokine receptor 2 (CCR2) pathway inhibitor or a pharmaceutically acceptable salt thereof;
ii. a diluent in a concentration from 30% to 70% w/w;
iii. a binder in a concentration from 2% to 5% w/w;
iv. a disintegrant in a concentration from 2% to 8% w/w; and
V. a lubricant in a concentration from 0.05% to 5% w/w.
   for the treatment, amelioration or prevention of a condition or disease.

A kit for the treatment, amelioration or prevention of a condition or disease comprising:
a) a composition comprising:
   i. at least one chemokine receptor 2 (CCR2) pathway inhibitor or a pharmaceutically acceptable salt thereof;
   ii. a diluent in a concentration from 30% to 70% w/w;
   iii. a binder in a concentration from 2% to 5% w/w;
   iv. a disintegrant in a concentration from 2% to 8% w/w; and
   v. a lubricant in a concentration from 0.05% to 5% w/w.
b) instructions for use.

### DETAILED DESCRIPTION OF THE INVENTION

### Pharmaceutical Compositions

The present invention provides a composition for the delivery of a chemokine receptor 2 (CCR2) pathway inhibitor; preferably the CCR2 pathway inhibitor is repagermanium or propagermanium.

A number of problems can develop as powder comprising active agents flows through equipment such as bins, chutes, and press hoppers. If the powder is cohesive, an “arch” or “rathole” may form, resulting in erratic flow or a cessation in flow. In addition, flooding or uncontrolled discharge may occur if an arch or rathole spontaneously collapses.

For the blending of active agents with small particle sizes with other excipients, the adhesion force between particles can significantly reduce the powder flowability by increasing inter-particle friction. Poor flow can cause insufficient mixing of the blends (content uniformity) and rat-holing or arching in the hopper of a tablet press (segregation issue), impacting both product quality and operation.

No flow: no flow from a bin/hopper is a common and significant solids-handling problem, especially when handling fine/milled active agents and high active loading blends. In production, it can result in problems, such as starving downstream equipment, production delays, and the requirement for frequent operator intervention to reinitiate flow. No flow can be due to either arching (sometime referred to as “bridging” or “plugging”) or ratholing (also referred to as “piping”).

Arching: can occur in a bin when the powder empties through a central flow outlet, but once the powder directly above the central flow outlet has moved through the outlet, a dome or arch of further powder remains above the void left by the powder that has already flowed out. Arching is influenced by the bin/hopper geometry and outlet size. This problem will arise if the material has sufficient cohesive strength. Generally, powder discharge will stop once the flow channel directly above the central flow outlet empties. However, the unsupported arch or dome of powder above the void may collapse, leading to intermittent flow and possibly flooding or uncontrolled discharge.

Ratholing: can occur in a bin when the powder empties through a central flow outlet, but the powder at the bin walls remains stagnant and leaves an empty hole (rathole) through the powder, starting at the central flow outlet. Ratholing is influenced by the bin/hopper geometry and outlet size the powder is fed through. Similar to the problem of arching, this problem will arise if the powder has sufficient cohesive strength. Generally, powder discharge will stop once the flow channel surrounding the central flow outlet empties. However, there may be further slips of the stagnant powder around the walls, leading to intermittent flow and possibly flooding or uncontrolled discharge.

Examples of CCR2 pathway inhibitors such as repagermanium and propagermanium are prone to clumping, leading to the creation of ratholes and/or arches. Without being held to any theory, this appears to be due to static build-up during manufacture of therapeutic compositions comprising CCR2 pathway inhibitors.

In order to avoid ratholing and arching during the manufacture of a CCR2 pathway inhibitor (for example repagermanium or propagermanium) dosage form, there is need for a diluent in the dosage form that will prevent the CCR2 pathway inhibitor from forming ratholes and arches.

### Diluent

Diluents act as inert fillers in pharmaceutical dosage forms to increase weight, improve content uniformity and enable accurate dosing of active agents. Diluents may also provide better encapsulation and tableting properties such as improved cohesion or promotion of ingredient flow.

Natural diluents include starch; hydrolyzed starch; partially pregelatinized starch; simple carbohydrates such as mannitol, lactose, dextrin, glucose, sucrose, sorbitol; polysaccharides such as cellulose, chitin. Certain inorganic salts are also optionally used as diluents, including calcium triphosphate, magnesium carbonate and sodium chloride. Some commercially available diluents are Fast-Flo@ and STA-Rx 1500®.

The present invention has found that the problems associated with poor component flow during manufacture of dosage forms comprising CCR2 pathway inhibitors can be overcome by the addition of a suitable diluent.

Preferably the diluent is present in the composition in a concentration from 30% to 70% w/w, from 35% to 65%, from 40% to 60%, from 45% to 60% or from 50% to 60%. For example, the composition may comprise 55% diluent w/w.

The diluent may be chosen from the list comprising microcrystalline cellulose (MCC), colloidal silicon dioxide (CSD) or silicified microcrystalline cellulose (SMCC).

MCC is a polymer composed of glucose units connected by a 1-4 beta glycosidic bond. Microcrystalline cellulose is a commonly used excipient in the pharmaceutical industry. It has excellent compressibility properties and is used in solid dose forms, such as tablets and capsules. Both tablets and capsules can be formed that are hard, but dissolve quickly.

CSD is also known as silicon dioxide, with the chemical formula SiO₂. It has many uses in capsule and tablet-making: a diluent, anti-caking agent, adsorbent, disintegrant, or glidant to allow powder to flow freely when capsules and tablets are processed. CSD appear to be biologically inert.

SMCC is a synergistic, intimate physical mixture of microcrystalline cellulose (MCC) and colloidal silicon dioxide (CSD), with the silicon dioxide located on the surface of the microcrystalline cellulose. An example of a suitable SMCC is PROSOLV® SMCC90 LM. It has improved compaction properties in both wet granulation and direct compression compared to conventional microcrystalline cellulose. Silicified microcrystalline cellulose appears to have beneficial properties for use in the formulation of powder-filled capsules. The properties of SMCC are advantageous when used in conjunction with a CCR2 pathway inhibitor (for example repagermanium or propagermanium).

Preferably the SMCC is present in the composition in a concentration from 30% to 70% w/w, from 35% to 65%, from 40% to 60%, from 45% to 60% or from 50% to 60%. For example, the composition may comprise 55% diluent w/w.

The composition of the present invention may therefore comprise:
i. at least one chemokine receptor 2 (CCR2) pathway inhibitor or a pharmaceutically acceptable salt thereof;
ii. a diluent that is SMCC in a concentration from 30% to 70% w/w;
iii. a binder in a concentration from 2% to 5% w/w;
iv. a disintegrant in a concentration from 2% to 8% w/w; and
v. a lubricant in a concentration from 0.05% to 5% w/w.

Preferably the diluent, for example SMCC, has an average particle size from 10 μm to 500 μm as determined by Laser Diffraction, more preferably 20 μm to 300 μm. For example, the diluent may have an average particle size of 90 μm.

Preferably the diluent, for example SMCC, has a low moisture content. Preferably the moisture content is less than 20%, less than 15% or more preferably less than 10%. In one example, the moisture content may be less than 6%, 5%, 4% 3% or 2%.

There is therefore provided a composition comprising:
i. at least one chemokine receptor 2 (CCR2) pathway inhibitor or a pharmaceutically acceptable salt thereof;
ii. a diluent in a concentration from 30% to 70% w/w;
iii. a binder in a concentration from 2% to 5% w/w;
iv. a disintegrant in a concentration from 2% to 8% w/w; and
V. a lubricant in a concentration from 0.05% to 5% w/w.

The present invention provides a composition comprising:
i. at least one chemokine receptor 2 (CCR2) pathway inhibitor or a pharmaceutically acceptable salt thereof which is a direct antagonist of CCR2;
ii. a diluent in a concentration from 30% to 70% w/w;
iii. a binder in a concentration from 2% to 5% w/w;
iv. a disintegrant in a concentration from 2% to 8% w/w; and
v. a lubricant in a concentration from 0.05% to 5% w/w.

In one preferred embodiment the chemokine receptor pathway inhibitor is selected from the group consisting of:
(i) antagonists of chemokine receptors or components of the chemokine receptor pathway other than the chemokine receptor;
(ii) inverse agonists of chemokine receptors or components of the chemokine receptor pathway other than the chemokine receptor;
(iii) negative allosteric modulators of chemokine receptors or components of the chemokine receptor pathway other than the chemokine receptor;

The present invention therefore provides a composition comprising:
i. at least one chemokine receptor 2 (CCR2) pathway inhibitor or a pharmaceutically acceptable salt thereof which inhibits a component of the CCR2 pathway other than the chemokine receptor;
ii. a diluent in a concentration from 30% to 70% w/w;
iii. a binder in a concentration from 2% to 5% w/w;
iv. a disintegrant in a concentration from 2% to 8% w/w; and
V. a lubricant in a concentration from 0.05% to 5% w/w.

A more specific example of a chemokine receptor pathway inhibitor which targets a component of the chemokine receptor pathway other than the chemokine receptor might be an agent which blocks pathways associated with MCP-1 induced migration, activation of monocytes and chemotactic migration. Such agents that might be targeted include the functioning of cellular structures involved in chemotaxis such as microfilaments, microtubules, and intermediate filaments, and surface proteins such as glycosylphosphatidylinositol (GPI)-anchored proteins, and more specifically CD55, CD59 and CD16.

In one preferred embodiment the chemokine receptor pathway inhibitor is an antagonist of a chemokine receptor. Known antagonists of chemokine receptors include: RS504393, RS102895 (Sigma-Aldrich); MLN-1202 (Millennium Pharmaceuticals); INCB3344, INCB3284 and INCB8696 (Incyte Pharmaceuticals); MK-0812 (Merck); CCX140, CCX872-B [CCX-872] (ChemoCentryx), PF-4136309, PF-04178903, PF-04634817 (Pfizer); BMS-741672 (Bristol-Myers Squibb); Cenicriviroc (Allergan). In one preferred embodiment the chemokine receptor pathway inhibitor is selected from the group comprising RS504393, RS102895, MLN-1202, INCB8696, MK-0812, CCX140, PF-4136309, BMS-741672.

In one preferred embodiment the CCR2 pathway inhibitor is an antagonist of chemokine receptor 2, such as propagermanium, repagermanium or CCX140. Propagermanium, repagermanium and CCX140 are prone to clumping, leading to the creation of ratholes and/or arches.

Preferably, the chemokine receptor pathway inhibitor is propagermanium or repagermanium. The INN for propagermanium is INN #6260 and repagermanium is INN #6259. Propagermanium has been used as an active agent against chronic hepatitis and has been shown to specifically inhibit in vitro chemotactic migration of monocytes by MCP-1 through a mechanism that seems to require glycosylphosphatidylinositol (GPI)-anchored proteins such as CD55, CD59 and CD16 (Yokochi, S. (2001) Journal of Interferon and Cytokine Research 21:389-398). Additional names for propagermanium and/or include 3-[(2-carboxyethyl-oxogermyl)oxy-oxogermyl] propanoic acid, repagermanium proxigermanium, Ge-132, bis (2-carboxyethylgermanium) sesquioxide (CEGS), 2-carboxyethylgermasesquioxane, SK-818, organic germanium, germanium sesquioxide, 3,3′-(1,3-dioxo-1,3-digermanoxanediyl)bispropionic acid, 3-oxygermylpropionic acid polymer and poly-trans-(2-carboxyethyl) germasesquioxane.

There is therefore provided a composition comprising:
i. repagermanium or propagermanium or a pharmaceutically acceptable salt thereof;
ii. a diluent in a concentration from 30% to 70% w/w;
iii. a binder in a concentration from 2% to 5% w/w;
iv. a disintegrant in a concentration from 2% to 8% w/w; and
V. a lubricant in a concentration from 0.05% to 5% w/w.

In one preferred embodiment the chemokine receptor pathway inhibitor inhibits the in vitro chemotactic migration of monocytes induced by MCP-1. In another preferred embodiment the chemokine receptor pathway inhibitor inhibits the in vitro chemotactic migration of monocytes induced by MCP-1 through a mechanism requiring glycosylphosphatidylinositol (GPI)-anchored proteins such as CD55, CD59 and CD16. In another preferred embodiment the chemokine receptor pathway inhibitor stabilizes the complexes CCR2/CD55 and/or CCR2/CD59 and/or CCR2/CD16. The chemokine receptor pathway inhibitor may be a peptide, polypeptide or small chemical entity. For example, the chemokine receptor pathway inhibitor may be a protein, binding protein or antibody.

The chemokine receptor pathway inhibitor may inhibit MCP-1 induced migration and activation of monocytes and chemotactic migration through the targeting of one or more glycosylphosphatidylinositol (GPI)-anchored proteins selected from the group comprising CD55, CD59 and CD16. The chemokine receptor pathway inhibitor may modify the complexes CCR2/CD55 and/or CCR2/CD59 and/or CCR2/CD16.

Propagermanium is a chemokine receptor pathway inhibitor, but it does not inhibit MCP-1 binding and appears to target glycosylphosphatidylinositol (GPI)-anchored proteins such as CD55, CD59 and CD16 and inhibits in-vitro chemotactic migration of monocytes by MCP-1 (Yokochi (2001); Yamada (2004) The Journal of Immunology 172:3869-3875).

The compositions of the present invention may comprise a range of excipients, both those listed below and others.

### Binders

Binders, compression aids and granulating agents bind the tablet ingredients together giving form and mechanical strength.

Materials used as binders include materials from natural products such as acacia, tragacanth, starch and gelatine. Other binders include methylcellulose (MC), ethyl cellulose (EC) and carboxymethyl cellulose (CMC), polyvinyl pyrrolidone (PVP; povidone) and hydroxypropylmethyl cellulose (HPMC; hypromellose). Some commercially available binders are Emdex®, Avicell®and Emcompress®.

Suitable binders for use in the compositions of the present invention include hypromellose (HPMC) and povidone (PVP). Hypromellose is a partly O-methylated and O-(2-hydroxypropylated) cellulose.

Preferably the concentration of binder in the compositions of the present invention is from 2% w/w to 5% w/w, from 2.5% to 5%, from 3% to 5%, from 3.5% to 5%. For example, the concentration of binder may be 4% w/w. An example of a suitable binder may be 4% w/w hypromellose (HPMC).

### Disintegrants

Disintegrants aid dispersion of the tablet in the gastrointestinal tract, releasing the active agent and increasing the surface area for dissolution.

Materials used as disintegrants include but are not limited to starch including the commercial disintegrant based on starch, Explotab®. Sodium starch glycolate, Amberlite™, sodium carboxymethylcellulose, ultramylopectin, sodium alginate, gelatine, orange peel, acid carboxymethyl cellulose, natural sponge and bentonite are also contemplated. Another form of the disintegrants is the insoluble cationic exchange resins. Powdered gums are also optionally used as disintegrants and as binders and these include powdered gums such as agar, karaya or tragacanth. Alginic acid and its sodium salt and crospovidone are also useful as disintegrants.

Suitable disintegrants for use in the compositions of the present invention include sodium starch glycolate and crospovidone. Sodium starch glycolate is the sodium salt of a carboxymethyl ether of starch or of a crosslinked carboxymethyl ether of starch. Disintegration occurs by rapid uptake of water followed by rapid and enormous swelling. Although the effectiveness of many disintegrants is affected by the presence of hydrophobic excipients such as lubricants, the disintegrant efficiency of sodium starch glycolate is unimpaired.

Preferably the concentration of disintegrant in the compositions of the present invention is from 2% w/w to 8% w/w, from 2% to 7%, from 3% to 6%, or from 4% to 6%. For example, the concentration of disintegrant may be 5% w/w. An example of a suitable disintegrant may be 5% w/w sodium starch glycolate.

### Lubricants/Glidants

In terms of powder flow, lubricants can improve the flowability of blends and aid unit operations by reducing the inter-particle friction. Lubricants may slow disintegration and dissolution. Glidants improve the flow of powders during tablet manufacturing by reducing friction and adhesion between particles and to aid rearrangement during compression. They may also be used as anti-caking agents. The properties of glidants and lubricants are somewhat different, although some compounds, such as starch and talc, may be used for both purposes.

Stearic acid and its salts, such as magnesium stearate and calcium stearate, are the most frequently used lubricants in the pharmaceutical industry. However, other lubricants such as polytetrafluoroethylene (PTFE), liquid paraffin, vegetable oils and waxes may be used. Exemplary soluble lubricants may also be used such as talc, starch, sodium lauryl sulfate, magnesium lauryl sulfate, sodium stearyl fumarate, polyethylene glycol of various molecular weights, and Carbowax® 4000 and 6000. The glidants may include starch, talc, pyrogenic silica and hydrated silicoaluminate.

Suitable lubricants for use in the compositions of the present invention include magnesium stearate and sodium stearyl fumarate. Magnesium stearate creates a less compact or dense (fluffy) finished product which is easier to fill into capsules. In one example, the lubricant is not sodium stearyl fumarate. Magnesium stearate is a compound of magnesium with a mixture of solid organic acids that consists chiefly of variable proportions of magnesium stearate (C₃₆H₇₀MgO₄) and magnesium palmitate (C₃₂H₆₂MgO₄).

Preferably the concentration of lubricant in the compositions of the present invention is from 0.05% w/w to 5% w/w, from 0.5% to 5%, from 1% to 5%, from 2% to 5%, from 3% to 5%, or from 4% to 5%. For example, the concentration of lubricant may be 5% w/w. An example of a suitable lubricant may be 5% w/w magnesium stearate.

TABLE 1
Excipients in exemplary
compositions of the present invention

| Ingredient | Function |
|---|---|
| API | Active agent |
| Silicified | Diluent |
| microcrystalline cellulose | |
| Lactose | Diluent |
| Mannitol | Diluent |
| Hypromellose | Binder |
| Povidone | Binder |
| Sodium starch glycolate | Disintegrant |
| Crospovidone | Disintegrant |
| Magnesium stearate | Lubricant |
| Sodium stearyl fumarate | Lubricant |

The CCR2 pathway inhibitor may be a pharmaceutically acceptable salt of the active agent. Pharmaceutically and veterinary acceptable salts include salts which retain the biological effectiveness and properties of the compounds of the present disclosure and which are not biologically or otherwise undesirable. In many cases, the compounds disclosed herein are capable of forming acid and/or base salts by virtue of the presence of amino and/or carboxyl groups or groups similar thereto. Acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as by way of example only, alkyl amines, dialkyl amines, trialkyl amines, substituted alkyl amines, di (substituted alkyl)amines, tri (substituted alkyl)amines, alkenyl amines, dialkenyl amines, trialkenyl amines, substituted alkenyl amines, di (substituted alkenyl)amines, tri (substituted alkenyl)amines, cycloalkyl amines, di(cycloalkyl)amines, tri (cycloalkyl)amines, substituted cycloalkyl amines, disubstituted cycloalkyl amines, trisubstituted cycloalkyl amines, cycloalkenyl amines, di(cycloalkenyl)amines, tri (cycloalkenyl)amines, substituted cycloalkenyl amines, disubstituted cycloalkenyl amines, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Pharmaceutically and veterinary acceptable acid addition salts may be prepared from inorganic and organic acids. The inorganic acids that can be used include, by way of example only, hydrochloric acid, hydrobromic acid, sulfuric acid, nitric acid, phosphoric acid, and the like. The organic acids that can be used include, by way of example only, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The pharmaceutically or veterinary acceptable salts of the compounds useful in the present disclosure can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences. 17th ed., Mack Publishing Company, Easton, Pa. (1985), p. 1418, the disclosure of which is hereby incorporated by reference. Examples of such acceptable salts are the iodide, acetate, phenyl acetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, y-hydroxybutyrate, β-hydroxybutyrate, butyne-1,4-dioate, hexyne-1,4-dioate, hexyne-1,6-dioate, caproate, caprylate, chloride, cinnamate, citrate, decanoate, formate, fumarate, glycollate, heptanoate, hippurate, lactate, malate, maleate, hydroxymaleate, malonate, mandelate, mesylate, nicotinate, isonicotinate, oxalate, nitrate, phthalate, terephthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzenesulfonate, p-bromophenylsulfonate, chlorobenzenesulfonate, propanesulfonate, ethanesulfonate, 2-hydroxyethanesulfonate, merhanesulfonate, naphthalene-1-sulfonate, naphthalene-2-sulfonate, p-toluenesulfonate, xylenesulfonate, tartarate, and the like.

TABLE 2
Exemplary compositions of the invention

| | % w/w | | | | | | | | |
|---|---|---|---|---|---|---|---|---|---|
| Ingredient | 1 | 2 | 3 | 4 | 5 | 6 | 7 | 8 | 9 |
| API | 50 | 50 | 35.3 | 35.3 | 35.3 | 35.3 | 35.3 | 35.3 | 35.3 |
| Silicified | | | 55.2 | 40.4 | 43.5 | 40.3 | 27.5 | | 46.4 |
| Microcrystalline | | | | | | | | | |
| Cellulose | | | | | | | | | |
| (SMCC90LM) | | | | | | | | | |
| Mannitol 200 | | | | | | 16.5 | 5.1 | 10.0 | 9.3 |
| SD | | | | | | | | | |
| Hypromellose | | | 4 | 15.8 | 4.4 | 3.7 | 3.4 | | |
| 2910-5 | | | | | | | | | |
| Povidone K29- | | | | | | | | | 3.8 |
| 32 1094R* | | | | | | | | | |
| Starch | | | 5 | 7.8 | 5 | 3.4 | 0.5 | | |
| Glycolate | | | | | | | | | |
| Sodium | | | | | | | | | |
| Crospovidone | | | | | 10.3 | | | | 4.8 |
| Magnesium | | 1 | 0.5 | 0.7 | 1.4 | | 0.5 | 0.7 | |
| Stearate | | | | | | | | | |
| Sodium stearyl | | | | | | 0.8 | | | 0.5 |
| fumarate NF* | | | | | | | | | |
| Lactose | 50 | 49 | | | | | 27.71 | 54.0 | |
| monohydrate | | | | | | | | | |
| TOTAL | 100 | 100 | 100 | 100 | 100 | 100.0 | 100.0 | 100.0 | 100.0 |
| CAPSULE SIZE | 3 | 2 | 0 | 0 | 0 | 0 | 0 | 0 | 0 |
| CAPSULE TYPE | Gelatin | Gelatin | HPMC | HPMC | HPMC | HPMC | HPMC | HPMC | HPMC |

### Method of Treatment

The present invention further provides a method for the treatment, amelioration or prevention of a condition or disease comprising administering to said subject a therapeutically effective amount of:
a) a composition comprising
   i. at least one chemokine receptor 2 (CCR2) pathway inhibitor or a pharmaceutically acceptable salt thereof;
   ii. a diluent in a concentration from 30% to 70% w/w;
   iii. a binder in a concentration from 2% to 5% w/w;
   iv. a disintegrant in a concentration from 2% to 8% w/w; and
   v. a lubricant in a concentration from 0.05% to 5% w/w.

The subject to be treated is preferably a mammal, including a human mammal.

The CCR2 pathway inhibitor may be administered alone, as a single therapy.

The CCR2 pathway inhibitor may be a pharmaceutically acceptable salt of the CCR2 pathway inhibitor.

### Condition or Disease

Generally, a range of ailments which are chemokine-related may be treated by the method of the present invention. This includes ailments that are related to increased or decreased production of chemokines, and/or increased or decreased responsiveness of cells to chemokines. A chemokine-related ailment should also be understood to mean a condition in which chemokine receptors display aberrant characteristics, are the target of a particular pathogen or are a target of a pharmacological intervention.

The ailments which are chemokine-related are inflammatory conditions, or conditions deriving from or associate with inflammation. The condition or disease to be treated or prevented may be chosen from: kidney disease, cardiovascular disease, eye disease, oncological disease, or pulmonary disease.

### Kidney Disease

Preferably, the condition or disease to be treated or prevented is a kidney disease. The kidney disease may be chosen from the list comprising: focal segmental glomerulosclerosis (FSGS; including idiopathic (primary) FSGS, secondary FSGS, genetic FSGS, and any other causes of FSGS), fibrotic disorders in the kidney, Immunoglobulin A nephropathy (IgAN), Alport's Syndrome, chronic kidney disease including chronic kidney disease caused by diabetic nephropathy, renal insufficiency (diabetic and non-diabetic), and renal failure conditions, including diabetic nephropathy, glomerulonephritis, scleroderma, glomerular sclerosis, proteinuria of primary renal disease and renal vascular hypertension.

### Cardiovascular Disease

Preferably, the condition or disease to be treated or prevented is a cardiovascular disease. The cardiovascular disease may be a cardiac disease, more preferably a coronary artery disease, preferably chosen from the list comprising: arthrosclerosis, pulmonary hypertension, ischaemic heart diseases, cardiomyopathies, and inflammatory heart diseases. The cardiovascular disease may be chronic heart failure, hypertension, congestive heart failure, left ventricular dysfunction, hypertrophic cardiomyopathy, diabetic cardiac myopathy, supraventricular and ventricular arrhythmias, atrial fibrillation, atrial flutter, myocardial infarction and its sequelae, atherosclerosis, angina, heart failure, angina pectoris, primary and pulmonary hypertension, vascular disorders.

### Eye Disease

Preferably, the condition or disease to be treated or prevented is an eye disease. For example, inflammatory and fibrotic diseases of the eye including anterior and posterior fibrotic disease including macular degeneration, vascular eye disease, and fibrovascular scarring and gliosis-related disease, and ischemic disease including diabetic retinopathy.

### Oncological Disease

Preferably, the condition or disease to be treated or prevented is an oncological disease. For example, solid tumours including but not limited to carcinoma, sarcoma and undifferentiated tumors, lymphoma, blastoma, glioma, melanoma, germ cell tumours, and hybrid tumours such as carcinosarcoma. The treatment may also include adjunct or combination treatment of the invention with one or more anti-oncologic treatments including but not limited to surgery, radiotherapies, hormone therapies, chemotherapies, immune-mediated therapies, cell-based therapies, and oncolytic viral therapies.

### Pulmonary Disease

Preferably, the condition or disease to be treated or prevented is a pulmonary (lung) disease. The pulmonary disease may be an infectious or non-infectious inflammatory lung disease, preferably chosen from the list comprising: community acquired pneumonia, chronic obstructive pulmonary disease, asthma, bronchiectasis, bronchiolitis, bronchitis, emphysema, pleurisy. Preferably the CCR2 pathway inhibitor is for use in the treatment, amelioration or prevention of Acute Respiratory Distress Syndrome (ARDS). ARDS is a type of respiratory failure characterized by rapid onset of widespread inflammation in the lungs.

The most common cause of ARDS is primary pneumonia, which can be bacterial, viral, or fungal. The second most common cause of lung injury is severe sepsis, which may be associated with pneumonia or a non-pulmonary infectious source, such as peritonitis. The other important major causes of ARDS include aspiration of gastric contents; haemorrhage and shock following major trauma; and several other less common causes such as severe acute pancreatitis, transfusion-associated lung injury, and drug reactions.

Recent experience has also found that ARDS may be found in subjects with infection by severe acute respiratory syndrome coronavirus 2 (SARS-CoV-2). Thus, the ARDS may be associated with infection by SARS-CoV-2.

Additional diseases to be treated or prevented include secondary aldosteronism, primary and secondary pulmonary hyperaldosteronism, insulin resistance, migraine, Raynaud's disease, luminal hyperplasia, cognitive dysfunction, Alzheimer's disease, stroke, hyperkalemia, preeclampsia, sarcoidosis, ischemia and reperfusion injury, atherogenesis, and rheumatoid arthritis.

The term “prevention” includes administration of the compositions of the present invention to subjects who have symptoms that indicate they are at risk of developing a kidney disease, cardiovascular disease, oncological disease, eye disease or pulmonary disease as indicated above, but may not, at the time of treatment, be diagnosed with the kidney disease, cardiovascular disease, eye disease or pulmonary disease. For example, the subject may have severe persistent cough and/or low oxygen levels, but not be diagnosed with ARDS. The subject may have elevated blood creatine or urinary protein, but not yet be diagnosed with kidney disease.

The term “amelioration” includes administration of the compositions of the present invention to subjects who have had a kidney disease, cardiovascular disease, oncological disease, eye disease or pulmonary disease and are at risk of developing post-disease conditions. For example, the disease may be ARDS and the post-ARDS conditions may be fibrosis and persistent inflammation. The post-ARDS conditions may be due to the ARDS itself or may be due to the use of ventilators during the acute phase of ARDS.

In one aspect, the chemokine receptor pathway inhibitor inhibits or partially inhibits a protein other than the chemokine receptor, more preferably, the pathway inhibitor is an agent which blocks MCP-1 induced migration and activation of monocytes and chemotactic migration through the targeting of glycosylphosphatidylinositol (GPI)-anchored proteins such as CD55, CD59 and CD16. Most preferably, the chemokine receptor pathway inhibitor is propagermanium or repagermanium.

### Measurement of Inhibition

The phrase “chemokine receptor 2 pathway inhibitor” is intended to include any compound or agent which inhibits or partially inhibits any one of the pathways associated with the chemokine receptor 2.

The term “inhibits” as used herein, means a reduction below detectable limits when compared to a reference. The phrase includes blocking, retarding, or impeding an action to prevent an undesirable result.

The term “partially inhibits” as used herein, means any reduction within detectable limits when compared to a reference. The phrase includes blocking, retarding, or impeding an action to prevent an undesirable result.

The inhibition or partial inhibition of the CCR2 pathway by the CCR2 pathway inhibitor may be measured using the in vitro methods set out herein, and include but are not limited to, biochemical or cellular assays for the assessment of in vitro chemotactic migration of CCR2-expressing neutrophils and other cells such as are known in the art, as well as measurement of inositol phosphate production, extracellular-regulated kinase (ERK) phosphorylation, CAMP production, actin-recruitment, protein phosphorylation, nuclear protein localization, gene transcription, label-free technologies (such as using impedance, light refraction or charge redistribution), G protein coupling using proximity reporter systems or other approaches, β-arrestin recruitment or mediated signalling, transcription factor-based reporter systems, microscopy visualization using fluorescent labels, use of antibodies to assess receptor cellular localization (such as enzyme-linked immunosorbent assays) and fluorescence-activated cell sorting.

The inhibition or partial inhibition of the CCR2 pathway by the CCR2 pathway inhibitor may be measured using the in vivo methods set out herein, and include but are not limited to, measurement of cellular and cytokine content of lung exudate, measurement of lung function including physical capacity of lung function using spirometry-based tests, or lung functional outputs measured using measurement blood gas or other biochemical measures, or improvement in functional benefit including clinical benefit measured by survival or quantitative methods such as walk tests or qualitative methods such as patient-reported outcome assessment. Inhibition or partial inhibition may be indicated by a qualitative improvement in lung structure as measured by one or more of the above-mentioned endpoints.

The inhibition or partial inhibition may be measured using the in vivo methods set out herein, and include but are not limited to, serial measurements of renal function made by the measurement of plasma creatinine and urea such as by way of an autoanalyser; the measurement of proteinuria, the measurement of albuminuria such as by way of a radioimmunoassay; and GFR (single shot isotopic technique); the assessment of endpoints such as renal and/or cardiac and/or ocular structure, by way of, for example, light microscopy (LM) for the assessment of glomerular and cardiac hypertrophy, glomerulosclerosis and/or fibrosis and/or podocyte change and/or; immunohistochemistry to measure the extent of matrix deposition and modulation of profibrotic growth factors and their activity; assessment of systolic blood pressure, modulation of insulin fasting plasma glucose, modulation of Hemoglobin A1c; and molecular biological techniques to assess renal and cardiac and ocular structure according to conventional assays such as known in the art. Inhibition or partial inhibition may be indicated by a qualitative improvement in renal and/or cardiac and/or ocular structure as measured by one or more of the above mentioned endpoints.

The term “component” as used herein in the context of a pharmaceutical composition of the invention, means the chemokine receptor pathway inhibitor.

### Dosage Forms and Compositions

The dosage form provided by the present invention may comprise a vial, cartridge, container, tablet or capsule comprising the pharmaceutical composition of the invention together with dosage instructions for the administration of the dosage form to a subject for the treatment, amelioration or prevention of a disease.

Compositions of the invention, in various aspects, may be administered by injection, or prepared for oral, pulmonary, nasal or for any other form of administration. Preferably the compositions are administered, for example, orally, intravenously, subcutaneously, intramuscularly, intraorbitally, ophthalmically, intraventricularly, intracranially, intracapsularly, intraspinally, intracisternally, intraperitoneally, buccal, rectally, vaginally, intranasally or by aerosol administration. Preferably the compositions of the invention are delivered via the oral, inhaled or topical route.

The mode of administration is in one aspect at least suitable for the form in which the composition has been prepared. The mode of administration for the most effective response may be determined empirically and the means of administration described below are given as examples, and do not limit the method of delivery of the composition of the present invention in any way. All the compositions provided are commonly used in the pharmaceutical industry and are commonly known to suitably qualified practitioners.

Preferably, the CCR2 pathway inhibitor is provided at a dose of from 10 mg to 500 mg per day, provided in one or more doses. Most preferably the CCR2 pathway inhibitor is provided at a dose of from 25 to 250 mg per day, provided in one or more doses. Preferably an adult dose is from 100 to 250 mg per day. Preferably a paediatric dose is from 25 to 125 mg per day.

It will be understood, however, that the specific dose level for any particular subject will depend upon a variety of factors including the activity of the specific compound employed, the age, body weight, general health, sex, diet, time of administration, route of administration, rate of excretion, combinations of drugs, and the severity of the particular condition or disease undergoing therapy.

### Excipients

The medicaments of the invention in certain aspects may include pharmaceutically acceptable nontoxic excipients and carriers. As used herein, a “pharmaceutical carrier” is a pharmaceutically acceptable solvent, suspending agent, excipient or vehicle for delivering the compounds to the subject. The carrier may be liquid or solid and is selected with the planned manner of administration in mind.

Contemplated for use herein are solid oral dosage forms, including tablets, capsules, pills, troches or lozenges, cachets or pellets. Solid dosage forms are described generally in Martin, Remington's Pharmaceutical Sciences, 18th Ed. (1990 Mack Publishing Co. Easton PA 18042) at Chapter 89, which is herein incorporated by reference. A description of possible solid dosage forms for the active agent is given by Marshall, in Modern Pharmaceutics (Chapter 10, Banker and Rhodes ed., (1979)). In general, the composition will include the compounds described as part of the invention (or a chemically modified form thereof), and inert ingredients which allow for protection against the stomach environment and release of the biologically active agent in the intestine.

Alternatively, the oral dosage form of the present invention may be a liquid, suspension or other appropriate dosage form. Such liquid oral dosage forms may be delivered directly into the feeding tube (nasogastric tube) of a patient who is in a coma and/or on a respirator and is unable to swallow a tablet etc. For example, the oral dosage form may be a capsule comprising a powder or small pellets, wherein the powder or small pellets may be dispensed from the capsule into a feeding tube, or dissolved into a liquid and dispensed into a feeding tube. Alternatively, the oral dosage form may be a powder or small pellets (optionally provided in a capsule) that can be dissolved in a liquid for administering to, for example children and the elderly who cannot swallow a tablet or capsule. For example, the oral dosage form comprising a CCR2 pathway inhibitor may be a powder or small pellets dissolved in a drink product such as water, apple juice or orange juice, or a food such as apple sauce. It is preferable that the solubilising medium be neutral or acidic in pH.

For pills, lozenges, moulded tablets or tablet triturates, moist massing techniques are contemplated. A coating or mixture of coatings can also be used on tablets, which are not intended for protection against the stomach. This includes sugar coatings, or coatings that make the tablet easier to swallow.

Exemplary capsules consist of a hard shell (such as gelatine) for delivery of dry active agents i.e. powder; for liquid forms, a soft gelatine shell may be used. The composition may be delivered in capsules. Preferably the capsules are gelatine capsules or vegetarian capsules such as VCaps® or Vcaps® plus. The capsules may be from size 4 to size 0. The capsules may contain the composition in the form of powders, granules or pellets.

For the CCR2 pathway inhibitor of the invention, the location of release may be the stomach, the small intestine (the duodenum, the jejunum, or the ileum), or the large intestine. One skilled in the art has available compositions that will not dissolve in the stomach yet will release the material in the duodenum or elsewhere in the intestine. In one aspect, the release will avoid the deleterious effects of the stomach environment, either by protection of the composition or by release of the compounds beyond the stomach environment, such as in the intestine.

To ensure full gastric resistance, a coating impermeable to at least pH 5.0 is used. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

The invention further provides a sustained release oral dosage form comprising a therapeutically effective pharmaceutical composition according to the invention, and a release retardant. Preferably such sustained release oral dosage forms are tablets or pills, or small granules or pellets.

As used herein, the term “sustained release” means the gradual but continuous or sustained release over a relatively extended period of the active agent content after oral ingestion. The release may continue after the pharmaceutical composition has passed from the stomach and through until and after the pharmaceutical composition reaches the intestine. The phrase “sustained release” also means delayed release wherein release of the active agent is not immediately initiated upon the pharmaceutical composition reaching the stomach but rather is delayed for a period of time, for example, until when the pharmaceutical composition reaches the intestine. Upon reaching the intestine, the increase in pH may then trigger release of the active agent from the pharmaceutical composition.

Though term “release retardant” is used herein, means a substance that reduces the rate of release of an active agent from a pharmaceutical composition when orally ingested. The release retardant may be a polymer or a non-polymer. The release retardant may be used according to any one of several sustained release systems including, for example, a diffusion system, a dissolution system and/or an osmotic system.

The release retardant may be an enteric coating or a semipermeable membrane. In one aspect of the present invention the release retardant is a water-soluble, water swellable and/or water insoluble polymer. In particular, water-soluble polymers are selected from the group comprising are ethylcellulose, hydroxypropyl cellulose, hydroxypropyl methyl cellulose or polyvinyl alcohol (PVA). In another aspect of the invention the release retardant is a non-polymeric release retardant such as hydrogenated castor oil or carnauba wax. The release retardant may be a mixture of a water-soluble polymer and a non-polymeric release retardant. One example of such a coating is a mixture of the water-soluble polymer PVA (for example Opadry®) and the non-polymeric release retardant carnauba wax.

The sustained release oral dosage form comprising a release retardant may be tablets or pills, or small granules or pellets.

Colourants and flavouring agents may optionally be included. For example, compounds may be formulated (such as by liposome or microsphere encapsulation) and then further contained within an edible product, such as a refrigerated beverage containing colorants and flavouring agents.

To aid dissolution of the active agent into the aqueous environment, a surfactant might be added in certain embodiments as a wetting agent. Surfactants may include, for example anionic detergents such as sodium lauryl sulfate, dioctyl sodium sulfosuccinate and dioctyl sodium sulfonate. Cationic detergents might be optionally used and could include benzalkonium chloride or benzethomium chloride. The list of potential nonionic detergents that could be included in the composition as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. When used, these surfactants could be present in the composition of the compounds either alone or as a mixture in different ratios.

Additives which potentially enhance uptake of the compounds are, for instance, the fatty acids oleic acid, linoleic acid and linolenic acid.

Controlled release composition may be desirable. The compositions are also contemplated. In certain aspects, the compounds could be incorporated into an inert matrix that permits release by either diffusion or leaching mechanisms i.e., gums. In some aspects, slowly degenerating matrices may also be incorporated into the composition. Another form of a controlled release of this active agent is by a method based on the Oros therapeutic system (Alza Corp.), i.e. the drug is enclosed in a semipermeable membrane which allows water to enter and push drug out through a single small opening due to osmotic effects. Some enteric coatings also have a delayed release effect.

In other aspects, a mix of materials might be used to provide a film coating. Film coating may be carried out, for example, in a pan coater or in a fluidized bed or by compression coating.

### Dosing Schedule

It will be appreciated that in certain aspects, the compositions of the invention may be given as a single dose schedule, or preferably, in a multiple dose schedule. A multiple dose schedule is one in which a primary course of delivery may be with 1 to 10 separate doses, is optionally followed by other doses given at subsequent time intervals required to maintain or reinforce the treatment. The dosage regimen will also, at least in part, be determined by the needs of the individual and the judgement of the practitioner.

The invention thus provides a tablet comprising the pharmaceutical composition of the invention; a capsule comprising the pharmaceutical composition of the invention, an injectable suspension comprising the pharmaceutical composition of the invention, and a composition for pulmonary delivery comprising the pharmaceutical composition of the invention.

### Use

The invention also provides for the use of:
i. at least one chemokine receptor 2 (CCR2) pathway inhibitor or a pharmaceutically acceptable salt thereof;
ii. a diluent in a concentration from 30% to 70% w/w;
iii. a binder in a concentration from 2% to 5% w/w;
iv. a disintegrant in a concentration from 2% to 8% w/w; and
v. a lubricant in a concentration from 0.05% to 5% w/w.
   in the manufacture of a medicament for the treatment, amelioration or prevention of a condition or disease.

The invention also provides for the use of a composition comprising:
i. at least one chemokine receptor 2 (CCR2) pathway inhibitor or a pharmaceutically acceptable salt thereof;
ii. a diluent in a concentration from 30% to 70% w/w;
iii. a binder in a concentration from 2% to 5% w/w;
iv. a disintegrant in a concentration from 2% to 8% w/w; and
v. a lubricant in a concentration from 0.05% to 5% w/w.
   for the treatment, amelioration or prevention of a condition or disease.

Preferably the composition comprises from 2.5% to 65% w/w of the CCR2 pathway inhibitor. Preferably the CCR2 pathway inhibitor is a direct antagonist of CCR2. More preferably the CCR2 pathway inhibitor is repagermanium or propagermanium or a pharmaceutically acceptable salt thereof.

Preferably the diluent is silicified microcrystalline cellulose (SMCC). Preferably composition comprises from 30% to 70% w/w SMCC.

Preferably the binder is hypromellose (HPMC). Preferably the composition comprises from 2% to 5% w/w HPMC.

Preferably the disintegrant is sodium starch glycolate. Preferably the composition comprises from 2% to 8% w/w sodium starch glycolate.

Preferably the lubricant is magnesium stearate. Preferably the composition comprises from 0.05% to 5% w/w magnesium stearate.

### Kits

The invention also provides a kit for the treatment, amelioration or prevention of a condition or disease comprising:
a) a composition comprising:
   i. at least one chemokine receptor 2 (CCR2) pathway inhibitor or a pharmaceutically acceptable salt thereof;
   ii. a diluent in a concentration from 30% to 70% w/w;
   iii. a binder in a concentration from 2% to 5% w/w;
   iv. a disintegrant in a concentration from 2% to 8% w/w; and
   v. a lubricant in a concentration from 0.05% to 5% w/w.
b) instructions for use.

The contents of the kit can be lyophilized, and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the composition may be applied to an affected area of the animal, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

### General

All publications, including patents and patent applications, cited herein, whether supra or infra, are hereby incorporated by reference in their entirety. However, publications mentioned herein are cited for the purpose of describing and disclosing the protocols, reagents and vectors that are reported in the publications and which may be used in connection with the invention. Nothing herein is to be construed as an admission that the invention is not entitled to antedate such disclosure by virtue of prior invention.

As used herein and in the appended claims, the singular forms “a,” “an,” and “the” include the plural unless the context clearly dictates otherwise. Thus, for example, a reference to “a protein” includes a plurality of such proteins, and a reference to “an analyte” is a reference to one or more analytes, and so forth.

Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The invention described herein may include one or more ranges of values (e.g. size, concentration etc.). A range of values will be understood to include all values within the range, including the values defining the range, and values adjacent to the range that lead to the same or substantially the same outcome as the values immediately adjacent to that value which defines the boundary to the range.

Throughout this specification, unless the context requires otherwise, the word “comprise” or variations, such as “comprises” or “comprising” will be understood to imply the inclusion of a stated integer, or group of integers, but not the exclusion of any other integers or group of integers.

### EXAMPLES

### Example 1—Exemplary Compositions

Exemplary formulations that could be used in treatment of a condition of disease include the following.

TABLE 3
Exemplary repagermanium compositions for chronic use

| | FORMULA % w/w | | | | |
|---|---|---|---|---|---|
| Commodity Name | A | B | C | D | E |
| REPAGERMANIUM | 30 | 34.3 | 36.9 | 40 | 35.3 |
| Silicified Microcrystalline | 60.5 | 56.2 | 53.6 | 50.5 | 55.2 |
| Cellulose (SMCC90LM) | | | | | |
| HPMC 2910-5 | 4 | 4 | 4 | 4 | 4 |
| Starch Glycolate Sodium | 5 | 5 | 5 | 5 | 5 |
| Magnesium Stearate | 0.5 | 0.5 | 0.5 | 0.5 | 0.5 |

TABLE 4
Repagermanium capsule compositions

| | FORMULA % w/w | | | | |
|---|---|---|---|---|---|
| Commodity Name | A | B | C | D | E |
| REPAGERMANIUM | 30 | 34.3 | 36.9 | 40 | 35.3 |
| Silicified Microcrystalline | 60.5 | 56.2 | 53.6 | 50.5 | 55.2 |
| Cellulose (SMCC90LM) | | | | | |
| HPMC 2910-5 | 4 | 4 | 4 | 4 | 4 |
| Starch Glycolate Sodium | 5 | 5 | 5 | 5 | 5 |
| Magnesium Stearate | 0.5 | 0.5 | 0.5 | 0.5 | 0.5 |

TABLE 5
Repagermanium capsule compositions

| | FORMULA % w/w | | | | | |
|---|---|---|---|---|---|---|
| Commodity Name | 1 | 2 | 3 | 4 | 5 | 6 |
| REPAGERMANIUM | 30 | 34.3 | 37.0 | 40.0 | 35.3 | 36.9 |
| Silicified Micro- | 40.3 | 42.2 | 53.6 | 50.5 | 33.1 | 48.5 |
| crystalline Cellulose | | | | | | |
| (SMCC90LM) | | | | | | |
| Mannitol | 20.2 | 14.1 | 0.0 | 0.0 | 22.1 | 5.0 |
| HPMC 2910-5 | | 2.0 | 4.0 | 4.0 | | 4.0 |
| Povidone | 4 | 2.0 | | | 4.0 | |
| Starch Glycolate Sodium | | | 2.5 | 3.3 | 5.0 | 5.0 |
| Crospovidone | 5 | 5.0 | 2.5 | 1.7 | | |
| Magnesium Stearate | 0.5 | | 0.5 | 0.5 | 0.5 | 0.5 |
| Sodium Stearyl Fumarate | | 0.5 | | | | |

TABLE 6
Exemplary repagermanium
compositions for acute use

| | FORMULA % w/w | | | |
|---|---|---|---|---|
| Commodity Name | 1 | 2 | 3 | 4 |
| REPAGERMANIUM | 50 | 50 | 50 | 48.8 |
| Silicified Microcrystalline | | 24.5 | 12.25 | 22.7 |
| Cellulose (SMCC90LM) | | | | |
| Lactose | 49 | 24.5 | 36.75 | 22.7 |
| Starch Glycolate Sodium | | | | 4.9 |
| Magnesium Stearate | 1 | 1 | | 1.0 |
| Sodium Stearyl Fumarate | | | 1 | |

TABLE 7
Exemplary propagermanium compositions for chronic use

| | FORMULA % w/w | | | | |
|---|---|---|---|---|---|
| Commodity Name | A | B | C | D | E |
| PROPAGERMANIUM | 30 | 34.3 | 36.9 | 40 | 35.3 |
| Silicified Microcrystalline | 60.5 | 56.2 | 53.6 | 50.5 | 55.2 |
| Cellulose (SMCC90LM) | | | | | |
| HPMC 2910-5 | 4 | 4 | 4 | 4 | 4 |
| Starch Glycolate Sodium | 5 | 5 | 5 | 5 | 5 |
| Magnesium Stearate | 0.5 | 0.5 | 0.5 | 0.5 | 0.5 |

TABLE 8
Propagermanium capsule compositions

| | FORMULA % w/w | | | | |
|---|---|---|---|---|---|
| Commodity Name | A | B | C | D | E |
| PROPAGERMANIUM | 30 | 34.3 | 36.9 | 40 | 35.3 |
| Silicified Microcrystalline | 60.5 | 56.2 | 53.6 | 50.5 | 55.2 |
| Cellulose (SMCC90LM) | | | | | |
| HPMC 2910-5 | 4 | 4 | 4 | 4 | 4 |
| Starch Glycolate Sodium | 5 | 5 | 5 | 5 | 5 |
| Magnesium Stearate | 0.5 | 0.5 | 0.5 | 0.5 | 0.5 |

TABLE 9
Propagermanium capsule compositions

| | FORMULA % w/w | | | | | |
|---|---|---|---|---|---|---|
| Commodity Name | 1 | 2 | 3 | 4 | 5 | 6 |
| PROPAGERMANIUM | 30 | 34.3 | 37.0 | 40.0 | 35.3 | 36.9 |
| Silicified Micro- | 40.3 | 42.2 | 53.6 | 50.5 | 33.1 | 48.5 |
| crystalline Cellulose | | | | | | |
| (SMCC90LM) | | | | | | |
| Mannitol | 20.2 | 14.1 | 0.0 | 0.0 | 22.1 | 5.0 |
| HPMC 2910-5 | | 2.0 | 4.0 | 4.0 | | 4.0 |
| Povidone | 4 | 2.0 | | | 4.0 | |
| Starch Glycolate Sodium | | | 2.5 | 3.3 | 5.0 | 5.0 |
| Crospovidone | 5 | 5.0 | 2.5 | 1.7 | | |
| Magnesium Stearate | 0.5 | | 0.5 | 0.5 | 0.5 | 0.5 |
| Sodium Stearyl Fumarate | | 0.5 | | | | |

TABLE 10
Exemplary propagermanium
compositions for acute use

| | FORMULA % w/w | | | |
|---|---|---|---|---|
| Commodity Name | 1 | 2 | 3 | 4 |
| PROPAGERMANIUM | 50 | 50 | 50 | 48.8 |
| Silicified Microcrystalline | | 24.5 | 12.25 | 22.7 |
| Cellulose (SMCC90LM) | | | | |
| Lactose | 49 | 24.5 | 36.75 | 22.7 |
| Starch Glycolate Sodium | | | | 4.9 |
| Magnesium Stearate | 1 | 1 | | 1.0 |
| Sodium Stearyl Fumarate | | | 1 | |

### Example 2—in Use Stability Study

An In Use stability study was performed to determine the reconstitution and dosage information for administration of repagermanium IR capsules via nasogastric feeding tube.

The objective of the study was to:
1. measure the stability of the repagermanium drug product composition once reconstituted in water for administration via a gastric tube,
2. determine if there is any residual repagermanium remaining in the medicine pot after the first 15 mL solution has been removed, and
3. identify if additional flushing of the nasogastric tube will be required to deliver the correct dose of repagermanium.

### Reagents and Materials

DMX-200 capsules

Repagermanium reference standard: Bis(2-carboxyethygermanium (IV) sesquioxide

Lactose Monohydrate Fast Flo

Magnesium Stearate

Sterile water

Enfit® oral syringe-35 mL Monoject™ by Covidien™

Tube type 1: Nasogastric feeding tube-Kangaroo™ by Covidien™ 8884720841 E 8Fr/CH (2.7 mm)×47″ (109 cm)

Tube type 2: Nasogastric feeding tube-Kangaroo™ by Covidien™ 8884721237 E 12Fr/CH (4.0 mm)×43″ (109 cm)

### Reconstitution in a Medical Pot

There were 6 experiments in total. Each experiment was performed in duplicate (except for micro tests).

TABLE 11
Reconstitution in a medical pot

| | Experiment No | | | | | |
|---|---|---|---|---|---|---|
| | 1 | 2 | 3 | 4 | 5 | 6 |
| | Test | | | | | |
| | | 10 | 60 | 120 | 180 | |
| | Initial | min- | min- | min- | min- | Nasogastric |
| | (T0) | utes | utes | utes | utes | feedtube |
| Appearance | X | X | X | X | X | X |
| Assay | X | X | X | X | X | X |
| Related | X | X | X | X | X | X |
| substances | | | | | | |
| pH | X | X | X | X | X | |
| TAMC | X | | | X | X | |
| TYMC | X | | | X | X | |
| Residual drug | X | | X | | | |

TABLE 12
Assay results for reconstitution in a medical pot (15 ml water)

| | | | % of dose | % of dose |
|---|---|---|---|---|
| | | | reconstituted | reconstituted |
| | Time | | in 15 ml water | in 15 ml water |
| Experiment | (minutes) | Sample | No rinse | One rinse (15 ml) |
| 1 | 0 | A | 27.78 | 34.7 |
| | | B | 25.10 | 28.8 |
| 2 | 10 | A | 54.13 | N/A |
| | | B | 53.75 | N/A |
| 3 | 60 | A | 61.48 | 67.8 |
| | | B | 61.99 | 69.0 |
| 4 | 120 | A | 61.00 | N/A |
| | | B | 62.53 | N/A |
| 5 | 180 | A | 73.06 | N/A |
| | | B | 73.90 | N/A |
| 6 NG-1 | 0 | A | 30.47 | 39.4 |
| | | B | 19.18 | 24.1 |
| 6 NG-2 | 0 | A | 25.73 | 33.5 |
| | | B | 22.73 | 28.6 |

The results from the study showed that although the drug product was stable both chemically and microbiologically when reconstituted in 15 ml water for up to 3 h, the total amount of dose measured was less than the expected level of ≥90% repagermanium even after rinsing once with 15 ml. Study also showed that the reconstituted drug product did not block, interact with or adhere to nasogastric tubes with diameters of 2.7 mm and 4.0 mm.

### Example 3—Follow-Up in Use Stability Study

The objective of the study was to:
1. determine the appropriate HPLC sample preparation method to measure reconstituted repagermanium in the medical pot.
2. determine if a larger volume of water is required to reconstitute the repagermanium (2×15 ml rinse/wash).

Each experiment was performed in duplicate. During HPLC analysis of the samples, a control sample (Standard) was analysed in parallel.

TABLE 12
Reconstitution of
repagermanium capsules

| Experiment No | 1 |
|---|---|
| Test | 15 mL + 2 washes |
| | (15 ml each) |
| Appearance | X |
| Assay | X |
| Related substances | X |
| pH | X |
| Residual drug | X |

### Results Summary

Appearance of solution were recorded as “An opaque white suspension with undissolved particles”. Appearance of nasogastric tubes was recorded as “unchanged following administration”.

pH results were within acceptance criteria of pH 2 to pH 6.

Microbial load for each experiment was <10 cfu/g TAMC and TYMC and did not change from 0 minutes to 180 minutes.

TABLE 13
Assay results for reconstitution in a medical
pot (15 ml water) followed by 2 × 15 ml rinses

| | | | | | Assay | |
|---|---|---|---|---|---|---|
| | | | | | (reconstitution | |
| | | | Assay (mg) | | in 15 ml water) + | |
| | Time | | (reconstitution | % of | Rinse (2 × 15 | % of |
| Experiment | (minutes) | Sample | in 15 ml) | dose | ml) Total (mg) | dose |
| 1 | 0 | A | 95.67 | 79.73 | 110.33 | 91.9 |
| | | B | 91.98 | 76.65 | 110.43 | 92.0 |
| Control | N/A | N/A | 118.37 | N/A | N/A | 98.9 |

The results from the second study confirmed the conclusion from the first study, that there was loss of drug product during transfer for analysis by HPLC. Improved sample preparation for HPLC analysis confirmed reconstitution of the drug product in 15 mL water followed by a minimum of two 15 mL washes is sufficient to deliver ≥90% repagermanium when reconstituted in a medical pot.

The two studies demonstrated that repagermanium can be reconstituted in a medical pot with 15 mL of water and administrated via a nasogastric tube. Two 15 mL rinses of the medical pot with water are required to deliver an appropriate dose. When repagermanium is reconstituted in 15 ml of water in a medical pot and left to sit on the bench it remains chemically and microbiologically stable for up to 180 minutes.

### Example 4—Sustained-Release Repagermanium Tablet

The objective is to develop a stable tablet formulation with repagermanium as the active, to be sustained released over a period of 8 hours. Trials were performed to obtain a tablet which was capable of passing friability limits and required to be as small as possible to be aesthetically pleasing. The preferred manufacturing process is a dry blend direct compression process.

TABLE 15
Composition of coated sustained release repagermanium tablet

| INGREDIENTS | % w/w | |
|---|---|---|
| Active | | |
| Repagermanium | 8.0 | active |
| Excipients | | |
| Hypromellose | 30.7 | sustained-release |
| Cellulose-Microcrystalline | 9.4 | diluent/disintegrant |
| Calcium Hydrogen | 37.4 | diluent |
| Phosphate Dihydrate | | |
| Starch-Pregelatinised Maize | 9.4 | diluent/disintegrant |
| Silicon Doixide | 0.8 | glidant |
| Magnesium Stearate | 1.1 | lubricant |
| Coating | | |
| Opadry II 85G68918 | 3.2 | film forming |
| White/ARTG 11378 | | polymer & colourant |
| Carnauba Wax Powder | qs | processing Aid |
| Total weight | 100.00 | |

The tablet core contains 30 mg of repagermanium and is a round tablet 10.3 mm in diameter with a break-bar. The final tablet is film coated white.

The physical properties of the tablets were tested.

TABLE 16
Physical properties of tablets

| Batch | SD16-13-30A | |
|---|---|---|
| Batch size | 2000 | tablets |
| approximately | | |
| Hardness | 8.9 | kp |
| Thickness | 4.2 | mm |
| Friability | 0.6 | |
| Average tablet | 363 | mg |
| Weight (cores) | | |
| Average tablet | 370 | mg |
| Weight (coated) | | |

The tablet release profile was tested in 500 ml purified water.

TABLE 17
Tablet release profile

| | SD16-13-30A | |
|---|---|---|
| | % label | |
| Hours | claim (30 mg) | SPECIFICATION |
| 0 | 0.0 | |
| 0.5 | 7.2 | |
| 1 | 16.4 | NMT 30% of label claim |
| 2 | 31.3 | |
| 3 | 45.0 | NMT 60% of label claim |
| 4 | 56.1 | |
| 6 | 75.6 | NLT 60% of label claim |
| 7 | 83.4 | |
| 8 | 89.5 | NLT 80% of label claim |

### Example 5—Sustained-Release Propagermanium Tablet

The objective is to develop a stable propagermanium tablet formulation, to be sustained released over a period of 8 hours. The tablet passes friability limits and is as small as possible to be aesthetically pleasing. The preferred manufacturing process is a dry blend direct compression process.

TABLE 18
Composition of coated sustained
release repagermanium tablet

| INGREDIENTS | % w/w | |
|---|---|---|
| Active | | |
| Repagermanium | 8.0 | active |
| Excipients | | |
| Hypromellose | 30.7 | sustained-release |
| Cellulose-Microcrystalline | 9.4 | diluent/disintegrant |
| Calcium Hydrogen | 37.4 | diluent |
| Phosphate Dihydrate | | |
| Starch-Pregelatinised Maize | 9.4 | diluent/disintegrant |
| Silicon Doixide | 0.8 | glidant |
| Magnesium Stearate | 1.1 | lubricant |
| Coating | | |
| Opadry II 85G68918 | 3.2 | film forming |
| White/ARTG 11378 | | polymer & colourant |
| Carnauba Wax Powder | qs | processing aid |
| Total weight | 100.00 | |

The round 10.3 mm in diameter film-coated white tablet has a core containing 30 mg of propagermanium and has a break-bar.

1. A composition comprising:
i at least one chemokine receptor 2 (CCR2) pathway inhibitor or a pharmaceutically acceptable salt thereof;
ii. a diluent in a concentration from 30 to 70% w/w;
iii. a binder in a concentration from 2 to 5% w/w;
iv. a disintegrant in a concentration from 2 to 8% w/w; and
V. a lubricant in a concentration from 0.05 to 5% w/w.

2. The composition of claim 1 wherein the CCR2 pathway inhibitor is a direct antagonist of CCR2.

3. The composition of claim 1 wherein the CCR2 pathway inhibitor is repagermanium or propagermanium or a pharmaceutically acceptable salt thereof.

4. The composition of claim 1 wherein the CCR2 pathway inhibitor is provided in a concentration from 2.5% to 65% w/w.

5. The composition of claim 1 wherein the diluent is silicified microcrystalline cellulose (SMCC).

6. The composition of claim 1 wherein the binder is hypromellose (HPMC).

7. The composition of claim 1 wherein the disintegrant is sodium starch glycolate.

8. The composition of claim 1 wherein the lubricant is magnesium stearate.

9. A method for the treatment, amelioration or prevention of a condition or disease comprising administering to said subject a therapeutically effective amount of:
a) a composition comprising
i. at least one chemokine receptor 2 (CCR2) pathway inhibitor or a pharmaceutically acceptable salt thereof;
ii. a diluent in a concentration from 30 to 70% w/w;
iii. a binder in a concentration from 2 to 5% w/w;
iv. a disintegrant in a concentration from 2 to 8% w/w; and
v. a lubricant in a concentration from 0.05 to 5% w/w.

10. (canceled)

11. (canceled)

12. A kit for the treatment, amelioration or prevention of a condition or disease comprising:
a composition according to claim 1; and
b) instructions for use.

13. The method of claim 9, wherein the condition or disease to be treated, ameliorated or prevented is chosen from: kidney disease, cardiovascular disease, eye disease, oncological disease, or pulmonary disease.

14. The method, use or kit of claim 13 wherein:
i. the kidney disease is focal segmental glomerulosclerosis (FSGS) or chronic kidney disease caused by diabetic nephropathy;
ii. the pulmonary disease is Acute Respiratory Distress Syndrome (ARDS).

15. The method of claim 9, wherein the CCR2 pathway inhibitor is provided at from 10 mg to 500 mg per day, provided in one or more doses.

16. The method of claim 9, wherein the CCR2 pathway inhibitor is a direct antagonist of CCR2.

17. The method of claim 9, wherein the CCR2 pathway inhibitor is repagermanium or propagermanium or a pharmaceutically acceptable salt thereof.

18. The method of claim 9, wherein the CCR2 pathway inhibitor is provided in a concentration from 2.5% to 65% w/w.

19. The method of claim 9, wherein the diluent is silicified microcrystalline cellulose (SMCC).

20. The method of claim 9, wherein the binder is hypromellose (HPMC).

21. The method of claim 9, wherein the disintegrant is sodium starch glycolate.

22. The method of claim 9, wherein the lubricant is magnesium stearate.

23. A composition comprising:
i. at least one chemokine receptor 2 (CCR2) pathway inhibitor or a pharmaceutically acceptable salt thereof;
ii. a diluent that is silicified microcrystalline cellulose (SMCC) in a concentration from 30 to 70% w/w;
iii. a binder that is hypromellose (HPMC) in a concentration from 2 to 5% w/w;
iv. a disintegrant that is sodium starch glycolate in a concentration from 2 to 8% w/w; and
V. a lubricant that is magnesium stearate in a concentration from 0.05 to 5% w/w.

24. The composition of claim 1 for oral capsule delivery.

25. The composition of claim 1 for nasogastric delivery.
